(19) United States
(12) Patent Application Publication
(10) Pub. No.: US 2005/0245852 A1
(43) Pub. Date: Nov. 3, 2005

(54) System and method for providing therapy to an area of a body

(76) Inventors: Kimberly L. Ellefson, Oshkosh, WI (US); MeeWha Lee, Appleton, WI (US); James D. Milner, Appleton, WI (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/835,938

(22) Filed: Apr. 30, 2004

(51) Int. Cl. A61F 5/00; A61N 1/36; A61N 1/38

(52) U.S. Cl. 602/2

(57) ABSTRACT

In some example embodiments, the present invention relates to a system for providing therapy to a body. The system includes a first patch that applies a first therapy to the body when the first patch is placed near the body. The first patch is customized to fit on a first area of the body. The system further includes a second patch that applies a second therapy to the body when the second patch is placed near the body. The second patch may be a different size than the first patch such that the second patch is customized to fit on a second area of the body and is detachably secured to the first patch to form a third patch that is a combination of the first patch and the second patch. In some embodiments, the first therapy may be different than the second therapy.

### FIELD

Some embodiments of this invention relate to a system and method for providing therapy to a body, and in particular to a system and method that include placing a patch on a body in order to provide therapy to an area of the body.

### BACKGROUND

Applying therapy to various parts of a body is a recognized practice for curing or alleviating multiple kinds of physical problems. One example therapy involves cooling an area of a body by applying ice packs near the area of the body in an effort to reduce swelling, inflammation and/or muscle pain. Another example therapy applies heat to an area of a body using a heating pad to treat symptoms such as stiffness, muscle pain, cold hands and feet, lumbago, rheumatism and neuralgia (among others). Other therapies include applying an analgesic (for pain) or an antibiotic (for infection) to the area of the body.

One concern with using heating pads and ice bags to apply therapy is that they are difficult to attach to an area of a body because they need to be held in place by the injured person or someone assisting the injured person. Holding a heating pad or ice bag against an injured area of a body can cause patient discomfort because a relatively large force is typically required in order to keep the ice bag or heating pad in place.

Another method of cooling or heating an area of a body includes applying hot or cold patches against the area of the body. The patch is typically attached directly, or indirectly, to the area of the body using an adhesive. An analgesic and/or antibiotic may also be incorporated into such a patch in order to treat the area for pain and/or infection.

A typical cold patch generates cooling via an endothermic reaction that takes place within the cold patch. The cold patch usually includes a solute and a liquid that are both stored within a common enclosure. The liquid and solute are initially segregated from another within the enclosure and then mixed within the enclosure to form an endothermic solution that reduces the temperature of the cold patch.

A typical heat patch generates heat via an exothermic reaction that takes place within the heat patch. Heat patches that generate heat using an exothermic reaction usually include an enclosure and a heating composition which is stored within the enclosure. At least one section of the enclosure is air-permeable such that exposing the heating composition to air generates an exothermic reaction that increases the temperature of the heat patch.

One drawback with using patches to apply therapy is that they are typically made in certain sizes and shapes. Therefore, a particular patch may be suitable for applying therapy to some areas of a body but not other areas of the body.

There is a need for a system and method that allow a user or therapist to conveniently provide therapy to an area of a body by applying an appropriately sized patch to the area of the body. The system and method should also be capable of allowing a user or therapist to apply an appropriately sized patch to more than one area of the body.

### SUMMARY OF THE INVENTION

The present invention relates to a system and method for providing therapy to a body. The system and method include placing a patch on an area of a body in order to provide therapy to the area of the body. The system and method provide a user or therapist with the flexibility to apply an appropriately sized patch to the area of the body. The system and method are also effective at applying an appropriately sized patch to more than one area of the body.

In some example embodiments, the present invention relates to a system for providing therapy to a body. The system includes a first patch that applies a first therapy to the body when the first patch is placed near the body. The system further includes a second patch that applies a second therapy to the body when the second patch is placed near the body. The second patch is detachably secured to the first patch to form a third patch that is a combination of the first patch and the second patch.

In other example embodiments, the present invention relates to a method of providing therapy to a body. The method includes selecting one of a first patch, a second patch or a third patch from a system for providing therapy to the body where the second patch is detachably secured to the first patch to form the third patch. The method further includes applying the selected patch near an injured area of the body.

In still other example embodiments, the present invention relates to a system for providing therapy to a body. The system includes a first patch that applies a first therapy to the body when the first patch is placed near the body. The first patch is customized to fit on a first area of the body. The system further includes a second patch that applies a second therapy to the body when the second patch is placed near the body. The second patch is customized to fit on a second area of the body and is detachably secured to the first patch to fonn a third patch that is a combination of the first patch and the second patch.

The purposes and features of the present invention will be set forth in the description that follows. Additional features of the invention will be realized and attained by the product and processes particularly pointed out in the written description and claims hereof, as well as from the appended drawings.

### BRIEF DESCRIPTION OF THE DRAWINGS

The present invention will be more fully understood, and further features will become apparent, when reference is made to the following detailed description and the accompanying drawings. The drawings are merely representative and are not intended to limit the scope of the claims. Like parts depicted in the drawings are referred to by the same reference numerals.

FIG. 1 is a plan view of an example system for providing therapy to a body.

FIG. 2 is a plan view of the system shown in FIG. 1 as a second patch in the system is being torn from a first patch.

FIG. 3 is a plan view illustrating the first and second patches of the system shown in FIGS. 1 and 2 mounted onto a body.

FIG. 4 is a plan view illustrating the third patch of the system shown in FIGS. 1 and 2 mounted onto a body.

FIG. 5 is a plan view illustrating an opposing side of the system shown in FIG. 1.

FIG. 6 is a plan view of another example system for providing therapy to a body.

FIG. 7 is a plan view of the system shown in FIG. 6 as a second patch in the system is being torn from a first patch.

FIG. 8 is a plan view illustrating the first and second patches of the system shown in FIGS. 6 and 7 mounted onto a body.

FIG. 9 is a plan view illustrating the third patch of the system shown in FIGS. 6 and 7 mounted onto a body.

### DETAILED DESCRIPTION OF THE INVENTION

In the following detailed description, reference is made to the accompanying drawings, which show specific embodiments in which the invention may be practiced. These embodiments are described in sufficient detail to enable those skilled in the art to practice the invention. It is to be understood that other embodiments may be utilized and structural changes made, such that the following detailed description is not to be taken in a limiting sense.

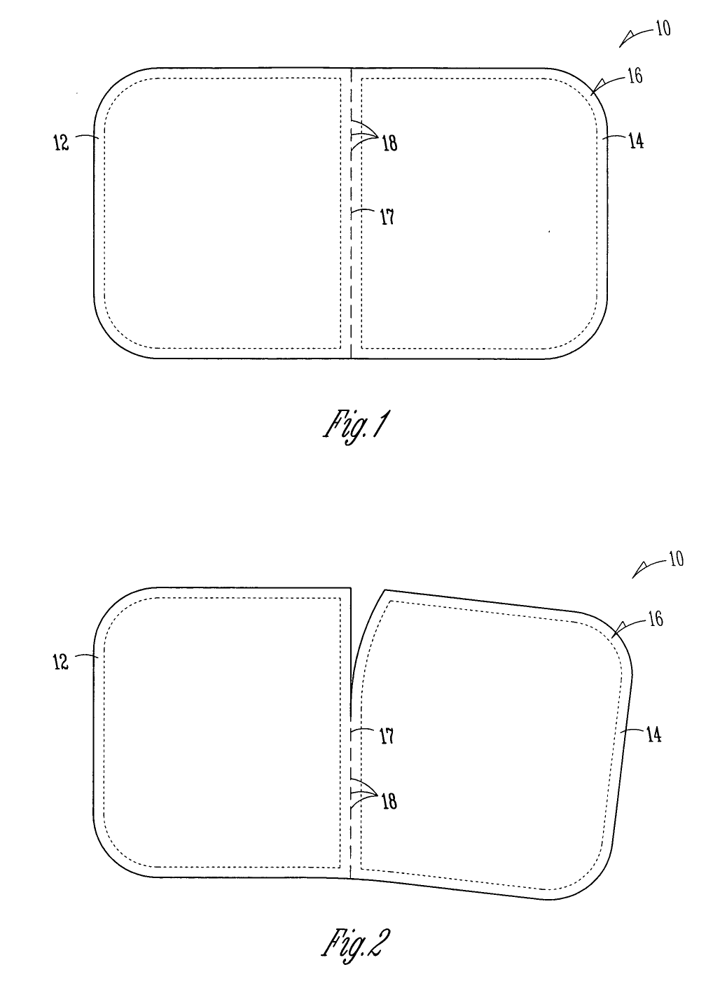

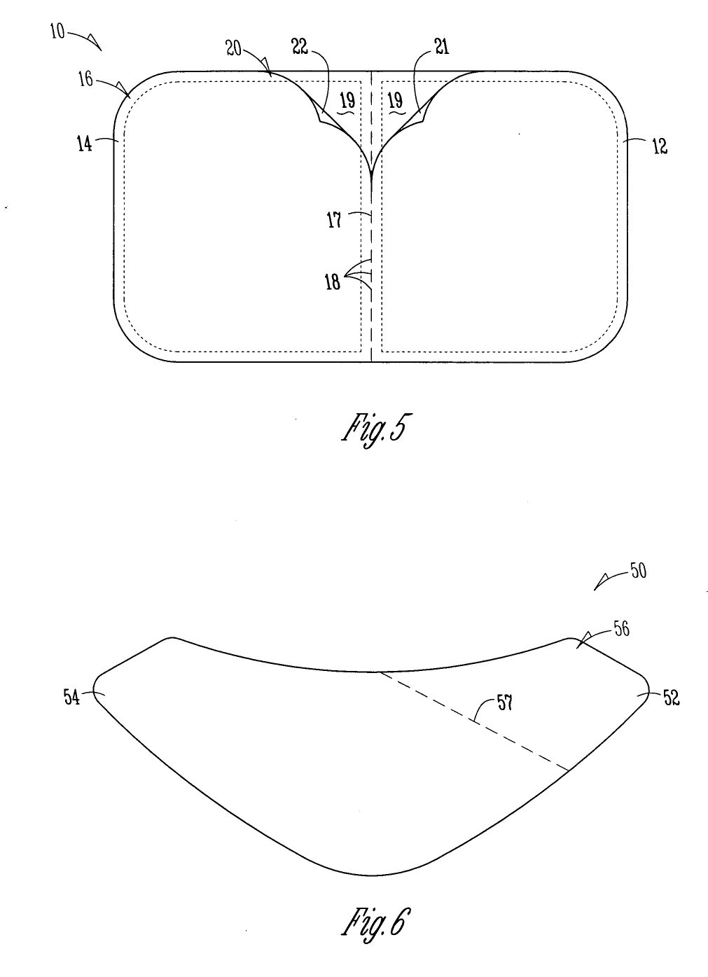

FIGS. 1-4 illustrates a system 10 for providing therapy to a body 100 (body 100 only shown in FIGS. 3-4). The system 10 includes a first patch 12 that applies a first therapy to the body 100 when the first patch 12 is placed near the body 100. The system further includes a second patch 14 that applies a second therapy to the body 100 when the second patch 14 is placed near the body 100. The second patch 14 is detachably secured to the first patch 12 to form a third patch 16 that is a combination of the first patch 12 and the second patch 14. In the illustrated example embodiment, the first patch 12 and the second patch 14 are the same size and shape.

As used herein, patch refers to any type of patch, pack, bag or pouch that may be used to apply therapy to a body. In addition, any of the first, second and third patches 12, 14, 16 may be capable of being attached directly, or indirectly, to the body 100.

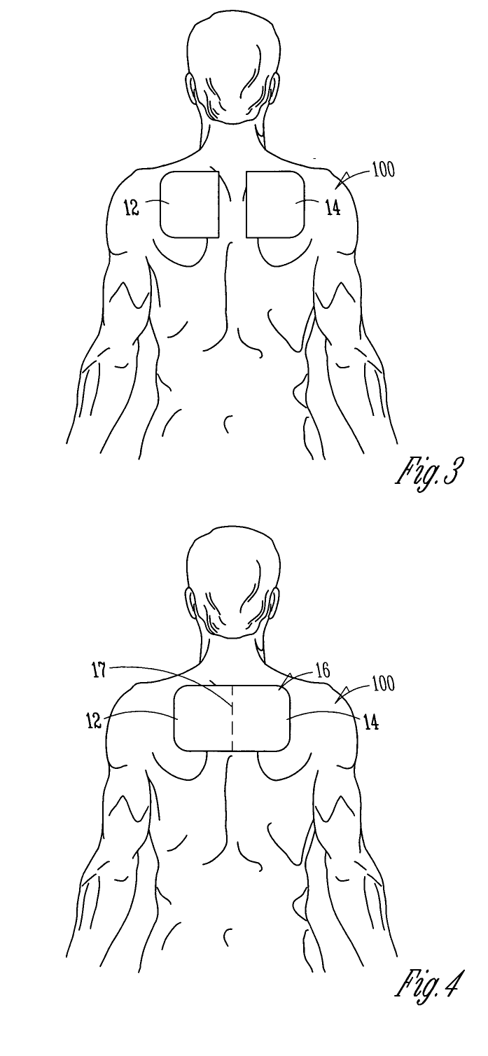

As shown in FIGS. 1-2, the first patch 12 and the second patch 14 may be detachably secured together by a perforated section 17 that is torn to separate the first patch 12 from the second patch 14 (FIG. 2). In some embodiments, the perforated section 17 is formed of slits 18 that are between the first and second patches 12, 14. Although slits 18 are shown in FIGS. 1-2, it should be noted that other types of perforations such as holes and indentations (among others) may be used to separate the first patch 12 and the second patch 14.

The slits 18 may be formed in the perforated section 17 by cutting between the first and second patches 12, 14. The ratio of the material that is cut to the material that is uncut within the perforated section 17 determines the size of the slits 18. As the total size of the slits 18 in the perforated section 17 increases, it becomes easier to tear apart the first patch 12 from the second patch 14.

In addition, when the third patch 16 is applied to an area of the body 100, the perforated section 17 may give the third patch 16 greater flexibility in the area of the perforated section 17. The increased flexibility of the third patch 16 at the perforated section 17 may allow the third patch 16 to fit more readily onto a highly contoured area of the body 100 (see, e.g., FIG. 4).

In some embodiments, the first and second patches 12, 14 may be heat patches that apply heat therapy to the body 100. In some forms, each of the heat patches may include an enclosure that has a gas-permeable section and a heating composition which is sealed inside the enclosure. The heating composition may be capable of generating heat when a gas, such as oxygen contained in ambient air, is received through the gas-permeable section of the enclosure.

When the first and second patches 12, 14 are heat patches they may be stored in a hermetic environment (e.g., a sealed bag) such that the heating composition remains inactive until the heat patches are removed from the hermetic environment. Once the heat patches are removed from the hermetic environment, the heating composition within each patch is exposed to air such that an exothermic reaction takes place within the heat patches. The exothermic reaction generates heat within the heat patches that increases the temperature of the heat patches.

Any conventional heating composition may be used to induce an exothermic reaction within the heat patches. Some example heating compositions include iron powder as the main active ingredient.

In other embodiments, the first and second patches 12, 14 may be cooling patches that apply cooling therapy to the body 100. The cooling patches may each include an enclosure that has a solute and a liquid sealed inside the enclosure. The solute and the liquid are initially segregated within each enclosure by a membrane. Rupturing the membrane in one or more the enclosures (e.g., by applying pressure to the enclosure) mixes the liquid with the solute to produce an endothermic solution within the enclosure.

Any conventional solute may be used to induce an endothermic reaction within the cold patches. One example solute includes ammonium nitrate.

It should be noted that in any of the patches shown, described and/or referenced herein, one or more of the patches may include a medication, such as a transdermal and/or topical medication that is applied to the body. Some example medications include topical analgesics and/or antibiotics.

In addition, one or more the patches may be treated with a variety of therapeutic additives, such as herbs, vitamins and/or botanicals. Another example therapeutic additive may include magnetic elements, such as particles chips, and/or disks. Some example botanicals include lavender, chamomile, moisturizers, lipids, essential oils and fragrances. The therapeutic additives could be inside the patches or sprayed directly on the patches.

In some embodiments, at least one of the first and second patches may also include non-steroidal anti-inflammatory drugs. Some example non-steroidal anti-inflammatory agents include aspirin, ibuprophen, ketoprophen and naproxen sodium (among others). In some forms, the non-steroidal anti-inflammatory agents may be drugs that are available by prescription only.

The first, second and third patches 12, 14, 16 may also be applied to an affected skin area to deliver treatment from various skin ailments. Some example ailments include irritation due to sun bums, heat bums, insect bites, athletes foot, dry skin, or other chronic skin conditions.

It should be noted that any combination of the therapies described herein may be part of the first and/or second patches 12, 14. As an example, at least one of the patches may be a heat patch that also includes a transdermal analgesic. In addition, the first and second patches may provide the same, or different, therapies to the body 100.

FIG. 5 shows an opposing side of the system 10 that is illustrated in FIGS. 1-2. In the illustrated example embodiment, the system 10 includes an adhesive 19 that is mounted on the first patch 12 and the second patch 14 in order to secure the first, second and third patches 12, 14, 16 to the body 100. The system 10 may further include a releasable cover 20 that is mounted on the adhesive 19 to protect the adhesive 19. In some embodiments, the releasable cover 20 may include a first section 21 that covers the adhesive 19 on the first patch 12 and a second section 22 that covers the adhesive 19 on the second patch 14. Depending on which of the first, second and/or third patches 12, 14, 16 are being attached to the body 100, one, or both, sections 21, 22 of the releasable cover 20 may be removed to expose the adhesive 19.

As examples, the adhesive 19 may be a styrene-isoprene-styrene copolymer, a silicone or a hydrogel, although any conventional adhesive may be used. In addition, the releasable cover 20 may be a polyethylene film, silicone paper or any other material that can be removed from the adhesive 19.

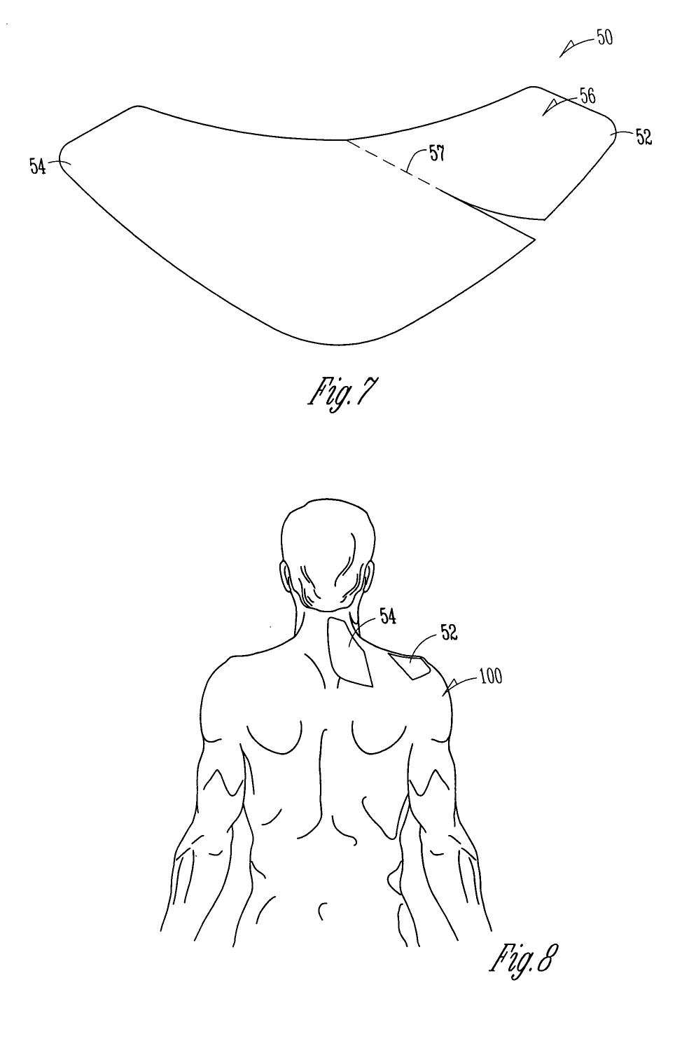

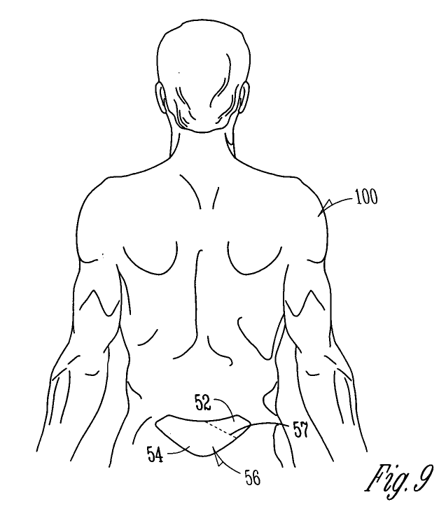

FIGS. 6-9 illustrate another example system 50 for providing therapy to a body 100 (body 100 only shown in FIGS. 8-9). The system 50 includes a first patch 52 that applies a first therapy to the body 100 when the first patch 52 is placed near the body 100. The system further includes a second patch 54 that applies a second therapy to the body 100 when the second patch 54 is placed near the body 100. The second patch 54 is detachably secured to the first patch 52 to form a third patch 56 that is a combination of the first patch 52 and the second patch 54.

FIG. 8 shows that the first patch 52 may be customized to fit on a first area (e.g., a shoulder) of the body 100 while the second patch 54 may be customized to fit on a second area (e.g., a neck) of the body 100. In addition, FIG. 9 shows that the third patch 56 may be customized to fit on a third area (e.g., lower back) of the body 100 such that the third patch 56 may be applied to the third area of the body 100 when the first and second patches 52, 54 are left together.

It should be noted that the first and second patches 52, 54 of the system 50 may be any of patches described above. In addition, the first and second patches 52, 54 may be detachably secured together by any type of perforated section 57 that may be torn to separate the first patch 52 from the second patch 54 (FIG. 7).

The proper size and shape of the first, second and third patches within each system 10, 50 will depend on the application where the systems 10, 50 are used. In some forms, one or more of the first, second and third patches may be long enough to fit around the arms, legs, head or torso of a body.

Although the systems are described herein include two patches that are detachably secured together, it should be noted that any number of patches may be detachably secured together to form a larger patch. In addition, the patches that form the larger patch may have any shape.

A method of providing therapy to a body 100 is described herein with reference to FIGS. 6-9. The method includes selecting one of a first patch 52, a second patch 54 or a third patch 56 from a system 50 for providing therapy to the body 100 where the second patch 54 is detachably secured to the first patch 52 to form the third patch 56. The method further includes applying the selected patch near an area of the body 100. FIG. 8 shows the selected patch as first patch 52 and/or second patch 54 while FIG. 9 shows the selected patch as third patch 56.

Selecting one of the first patch 52, the second patch 54 or the third patch 56 from the system 50 may include determining an appropriate size patch to apply to the area of the body 100. As an example, FIG. 9 shows that the third patch 56 was selected in order to provide therapy to a lower back of the body 100.

In some embodiments, applying the selected patch near the area of the body 100 may include applying the selected patch to the area of the body. As examples, FIGS. 8 and 9 show that the first, second and third patches 52, 54, 56 may be applied directly to the body 100, although in other embodiments the patches 52, 54, 56 may be applied onto clothing or a bandage that covers the area of the body 100. In addition, any of the first, second and/or third patches 52, 54, 56 may be held near the area of the body 100 using a bandage, elastic bandage, tape, wrap and/or elastic wrap (among other items).

It should be noted that selecting one of the first patch 52, the second patch 54 or the third patch 56 may include detaching the first patch 52 from the second patch 54 (e.g., by tearing a perforated section 57 between the first patch 52 and the second patch 54). In some embodiments, applying the selected patch near an area of the body 100 may include (i) applying at least one of the first patch 52 and the second patch 54 near the area of the body 100 when at least one of the first patch 52 and the second patch 54 is the selected patch; (ii) applying the third patch 56 near the area of the body 100 when the third patch 56 is the selected patch; and/or (iii) releasing a cover that protects an adhesive on the selected patch (see, e.g., adhesive 19 and cover 20 in FIG. 5), and adhering the selected patch near the area of the body 100 using the adhesive.

The operations discussed above with respect to the described methods may be performed in a different order from those described herein. In addition, FIGS. 1-9 are representational and are not necessarily drawn to scale. Certain proportions thereof may be exaggerated, while others may be minimized.

While the invention has been described in detail with respect to the specific aspects thereof, it will be appreciated that those skilled in the art, upon attaining an understanding of the foregoing, may readily conceive of alterations to, variations of, and equivalents to these aspects which fall within the spirit and scope of the present invention, which should be assessed accordingly to that of the appended claims.

1-31. (canceled)

32. A system for providing therapy to a body, the system comprising:
a first patch that applies a first therapy to the body when the first patch is placed near the body; and
a second patch that applies a second therapy to the body when the second patch is placed near the body, the second patch being detachably secured to the first patch to form a third patch that is a combination of the first patch and the second patch, the first therapy being different than the second therapy.

33. The system of claim 32 wherein the first and second patches are heat patches.

34. The system of claim 32 wherein the first and second patches are cooling patches.

35. The system of claim 32 wherein at least one of the first and second patches includes an antibiotic.

36. The system of claim 32 wherein at least one of the first and second patches includes an analgesic.

37. The system of claim 32 wherein at least one of the first and second patches includes an anti-inflammatory agent.

38. The system of claim 32 wherein at least one of the first patch and the second patch includes a magnetic element.

39. The system of claim 32 wherein the first patch and the second patch are detachably secured together by a perforated section that is torn to separate the first patch from the second patch.

40. The system of claim 32 further comprising an adhesive that is mounted on the first patch and the second patch to secure the first, second and third patches to the body.

41. A system for providing therapy to a body, the system comprising:
a first patch that applies a first therapy to the body when the first patch is placed near the body; and
a second patch that applies a second therapy to the body when the second patch is placed near the body, the second patch being detachably secured to the first patch to form a third patch that is a combination of the first patch and the second patch, the first patch being a different size than the second patch such that the first patch is customized to fit on a first area of the body and the second patch is customized to fit on a second area of the body.

42. The system of claim 41 wherein the first and second patches are heat patches.

43. The system of claim 41 wherein the first and second patches are cooling patches.

44. The system of claim 41 wherein the third patch is customized to fit on a third area of the body.

45. The system of claim 41 wherein the first patch and the second patch are detachably secured together by a perforated section that is torn to separate the first patch from the second patch.

46. The system of claim 41 further comprising an adhesive that is mounted on the first patch and the second patch to secure the first, second and third patches to the body.

47. The system of claim 46 further comprising a releasable cover that is mounted on the adhesive, wherein the releasable cover is removed to expose the adhesive.

48. The system of claim 41 wherein at least one of the first and second patches includes an anti-inflammatory agent.

49. A method of providing therapy to a body, the method comprising:
selecting one of a first patch, a second patch or a third patch from a system for providing therapy to the body where the second patch is detachably secured to the first patch to form the third patch, the first patch being customized to fit on a first area of the body, the first patch being a different size than the second patch such that the second patch is customized to fit on a second area of the body and the third patch is customized to fit on a third area of the body; and
applying the selected patch near an area of the body.

50. The method of claim 49 wherein applying the selected patch near the area of the body includes applying the selected patch to the area of the body.

51. The method of claim 49 wherein selecting one of the first patch, the second patch or the third patch includes detaching the first patch from the second patch.

52. The method of claim 51 wherein detaching the first patch from the second patch includes tearing a perforated section between the first patch and the second patch.

53. The method of claim 49 wherein applying the selected patch near an area of the body includes releasing a cover that protects an adhesive on the selected patch and adhering the selected patch near the area of the body using the adhesive.

54. The method of claim 49 wherein selecting one of the first patch, the second patch or the third patch includes determining an appropriate size patch to apply near an area of the body.

55. The method of claim 49 wherein applying the selected patch near an area of the body includes applying at least one of the first patch and the second patch near the area of the body when at least one of the first patch and the second patch is the selected patch.

56. A method of providing therapy to a body, the method comprising:
selecting one of a first patch, a second patch or a third patch from a system for providing therapy to the body where the second patch is detachably secured to the first patch to form the third patch, the first patch being adapted to provide a first therapy and the second patch being adapted to provide a second therapy that is different from the first therapy; and
applying the selected patch near an area of the body.

57. The method of claim 56 wherein applying the selected patch near the area of the body includes applying the selected patch to the area of the body.

58. The method of claim 56 wherein selecting one of the first patch, the second patch or the third patch includes detaching the first patch from the second patch.

59. The method of claim 58 wherein detaching the first patch from the second patch includes tearing a perforated section between the first patch and the second patch.

60. The method of claim 56 wherein applying the selected patch near an area of the body includes applying the third patch near the area of the body when the third patch is the selected patch.

61. The method of claim 56 wherein selecting one of the first patch, the second patch or the third patch includes determining an appropriate size patch to apply near an area of the body.

62. The method of claim 56 wherein applying the selected patch near an area of the body includes releasing a cover that protects an adhesive on the selected patch and adhering the selected patch near the area of the body using the adhesive.
